(19) United States
(12) Patent Application Publication
(10) Pub. No.: US 2007/0078390 A1
(43) Pub. Date: Apr. 5, 2007

(54) Injection syringe

(75) Inventor: Hong Cing-hong, Hsinchu (TW)

Correspondence Address: TROXELL LAW OFFICE PLLC, SUITE 1404, 5205 LEESBURG PIKE, FALLS CHURCH, VA, 22041, US

(73) Assignee: U-CONN TECHNOLOGY INC.

(21) Appl. No.: 11/215,029

(22) Filed: Aug. 31, 2005

(51) Int. Cl. A61M 5/00

(52) U.S. Cl. 604/110

(57) ABSTRACT

An injection syringe is used to assemble a needle thereon. The injection syringe has a syringe body, a needle base assembled inside the syringe body, and a pusher for pushing medicine. The syringe body forms first protrusions proximate an end and on an inner surface thereof. The needle base forms second protrusions on an outer surface thereof for cooperating with the first protrusions to engage or disengage the syringe body and the needle base. The needle base includes a needle connecting portion for connecting the needle, and a bugle-shaped bugle portion expands far away from the needle connecting portion. At least two ribs are formed on an inner surface of the bugle portion. A flange is formed at an end of the bugle portion. The pusher forms guiding posts, each guiding post having an inclined surface at an end thereof for abutting the flange. At least an abutting projection is formed on a top end of the pusher for biasing against the bugle portion.

### BACKGROUND OF THE INVENTION

1. Field of the Invention

The present invention relates to an injection syringe, and particularly relates to an injection syringe which rotates right/left to destroy an injection needle therein after injection.

2. Related Art

In order to avoid re-use or unexpected hurt to nurses, a conventional injection syringe often forms an opening member on rear of a needle base, and a conic holding member on a front of a pusher for telescopically fitting to the opening member after injection. The pusher retracts the needle and the needle base, and the holding member fits the opening member, thereby assuring safe use. It is necessary that the holding member fits the opening member tightly, and otherwise, the needle cannot be retracted successfully. This brings up some difficulty for users. Patients may feel pain if the holding member fits the opening member relatively tightly; while if the holding member fits the opening member relatively loosely, the pusher tends to retract the needle during filling medicine into the injection syringe, and therefore the injection syringe cannot be used. Another conventional injection syringe is designed in a way that the pusher is retracted to fit the holding member and the opening member after injection, but medicine remaining in the injection syringe often ejects out, polluting ambient environment.

To overcome defects mentioned above, another conventional injection syringe replaces the fitting structure with locking structure. L-shaped grooves are defined in an inner wall of a front of the syringe body, and a springy rod is assembled on a front of the pusher. A connecting base connects with the springy rod. A plurality of protrusions is formed around the connecting base for embedding into the L-shaped grooves. Before injection, the pusher rotates to embed the protrusions into the L-shaped grooves. After injection, the pusher rotates reversely to disengage the protrusions from the L-shaped grooves. The connecting base and the needle retract into the syringe body. The springy rod deflects slightly and biases against the connecting base. The pusher pushes the needle in abutment with an inner wall of the syringe body, destroying the needle.

However, the convention injection syringe has following deficiencies yet.

The needle can't be retracted smoothly. The protrusions lock or disengage from the L-shaped grooves in one direction. Correspondingly, the pusher has to rotate in one direction to draw out. Once the pusher rotates in a reverse direction, tending to rupture and fail to draw out.

The retracted needle deflects unreliably. The needle is retracted by the springy rod which is deflected slightly. The springy rod moves in narrow space, whereas pushing force thereof is limited, but weight of the needle and the connecting base is relatively large. The springy rod deflects at a small angle, and can't provide enough pushing force to bias against the retracted needle exactly. So the needle can't be destroyed as desired.

Manufacture process is complicated, and cost is high. The L-shaped grooves are defined in an inner wall of a front of a syringe body, and has to be formed integrally instead of being assembled as ordinarily. The shape and number of the L-shaped grooves increases cost and prevents mass production.

### SUMMARY OF THE INVENTION

Accordingly, an object of the present invention is to provide an injection syringe has double deflection to bias against a syringe body thereby destroying a needle thereof reliably.

Another object of the present invention is to provide an injection syringe which can be operated by one hand to destroy a needle thereof, thereby simplifying injection process.

The injection syringe of the present invention comprises a syringe body, a needle base, a pusher and a springy element. First protrusions are formed proximate an end of the syringe body and are distributed apart the same distance from each other. The needle base is mounted inside the syringe body and adjacent to the first protrusions. Second protrusions are distributed radially on an outer surface of the needle base for cooperating with the first protrusions. The number of the second protrusions is equal to that of the first protrusions. In assembly, second protrusions of the needle base are mounted on the first protrusions of the syringe body in a displaced position, and then rotate to a proper position. Similarly, in order to disengage the needle base from the syringe body, the needle base rotates left/right, and second protrusions of the needle base displaces with respect to the first protrusions of the syringe body to disengage.

### BRIEF DESCRIPTION OF THE DRAWINGS

FIG. 1 is an exploded view of an injection syringe of the present invention.

FIG. 2 is a cross-sectional view of a syringe body of the injection syringe.

FIG. 3 is a cross-sectional view of a needle base of the injection syringe.

FIG. 4 is a perspective view of a pusher of the injection syringe.

FIG. 5 is a cross-sectional view of the injection syringe before injection.

FIG. 6 is a cross-sectional view of the injection syringe after injection.

FIGS. 7-9 show the process of the needle base being drawn out.

FIGS. 10 and 11 are schematic views showing the needle being destroyed.

### DESCRIPTION OF THE PREFERRED EMBODIMENTS

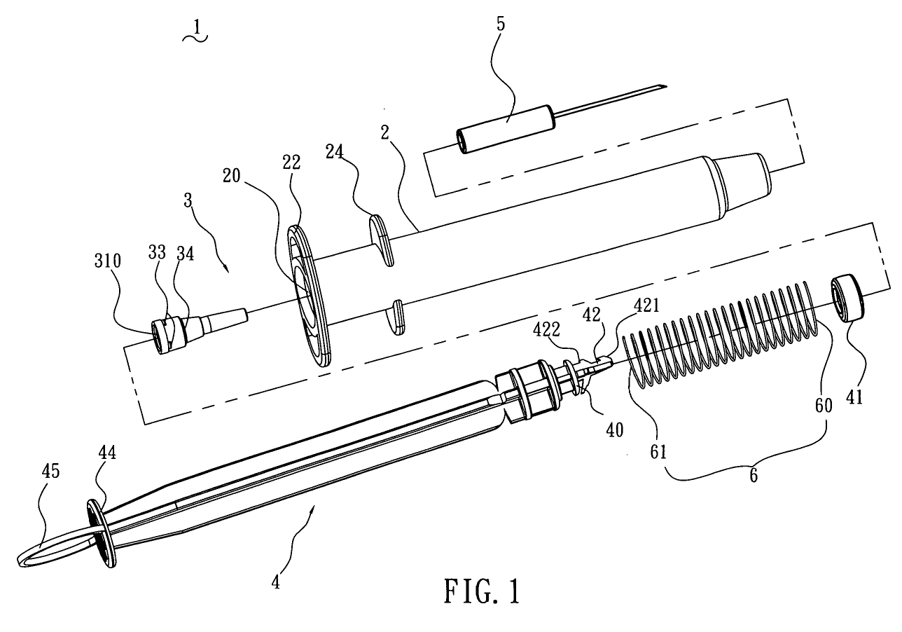

Referring to FIG. 1, an injection syringe 1 in accordance with the present invention is used to assemble a needle 5 thereon. The injection syringe 1 comprises a syringe body 2, a needle base 3, a pusher 4, and a springy element 6. The syringe body 2 is a hollow cylinder with two openings at opposite ends thereof, and defines a columned chamber 20 therethrough. Four first protrusions 21 are distributed on an inner surface of the syringe body 2 and are spaced radially apart the same distance from each other, for example, apart 90° therebetween. The first protrusions 21 are arcuate and proximate an end of the syringe body 2. A lip 22 is formed at an outer peripheral of an end of the syringe body 2 opposite to the first protrusions 21. A first stopper 23 and a second stopper 231 are integrally formed on an inner surface of the syringe body 2 and are respectively of ring shape. The first stopper 23 is located above the second stopper 231.

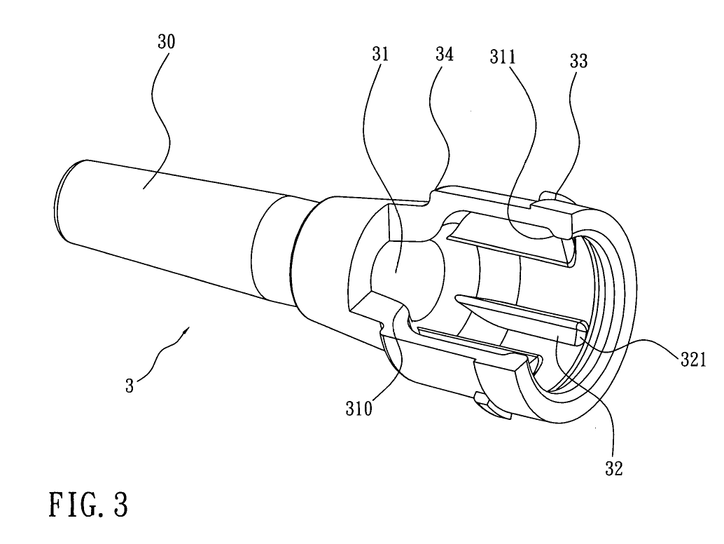

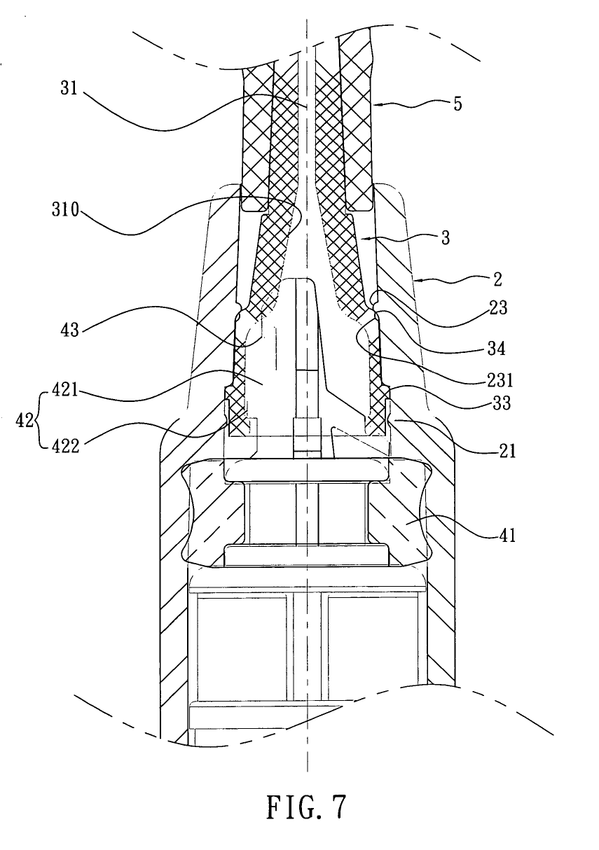

Referring to FIG. 3, the needle base 3 is mounted inside the syringe body 2 and adjacent to the first protrusions 21. The needle base 3 includes a needle connecting portion 30 for connecting a needle 5, and a passage 31 through two ends of the needle base 3. A bugle-shaped bugle portion 310 extends and expands far away from the needle connecting portion 30. Four ribs 32 are formed on an inner surface of the bugle portion 310 and are radially spaced apart 90° therebetween. Each rib 32 has a guiding surface 321. The bugle portion 310 forms a flange 311 inwardly at an end thereof. Second protrusions 33 are distributed on an outer surface of the needle base 3 for corresponding to the first protrusions 21, and are spaced radially apart the same distance from each other, for example, apart 90° therebetween. The number of the second protrusions 33 is equal to that of the first protrusions 21. A portion of the needle base 3 where the second protrusions 33 are formed has a cylindrical shape. In combination with FIG. 7, a shoulder 34 is formed on an outer surface of the needle base 3 and fits to the first stopper 23. As shown in FIG. 7, the shoulder 34 and the first stopper 23 partly overlap for anti-leaking. A ring-shaped recess (not labeled) is defined in a lower portion of the shoulder 34 for corresponding to the second stopper 231. In the case that the pusher 4 moves forward, the pusher 4 biases against the first stopper 23, preventing the pusher 4 from excess movement. In the case of retraction, the pusher 4 slides backward along the shoulder 34 with the assistance of the second stopper 231. In one embodiment of the present invention, the first stopper 23 and the second stopper 231 integrally protrude from the syringe body 2 for cooperating with the shoulder 34 and the recess to prevent the pusher 4 from excess movement and to facilitate the pusher 4 disengaging, simultaneously to prevent leakage.

Notably, as for an injection syringe with large diameter, the needle base 3 has an O-shaped leakproof ring (not shown) in the recess for corresponding to the second stopper 231, thereby further preventing leaking. As an injection syringe with small diameter, the O-shaped leakproof ring may be omitted.

In assembly, the needle base 3 is mounted onto the syringe body 2 in a displaced position, and then rotates to a proper position. Similarly, in order to disengage the needle base 3 from the syringe body 2, the needle base 3 rotates left/right, and the needle base 3 displaces with respect to the syringe body 2 to disengage.

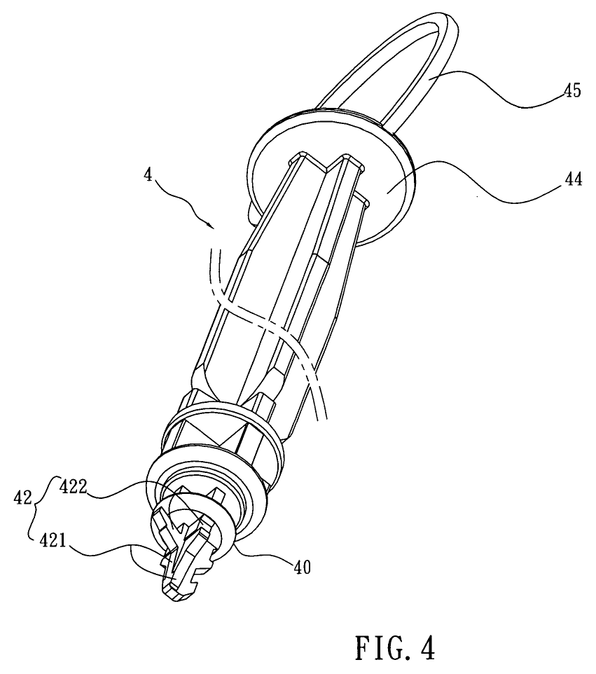

Referring to FIG. 4, the pusher 4 is cylindrical and is assembled inside the syringe body 2. The pusher 4 forms a neck 40 at an end thereof adjacent to the first protrusions 21. A leakproof gasket 41 (shown in FIG. 1) is provided above the neck 40. Four guiding posts 42 are formed above the neck 40 and are radially spaced 90° apart therebetween. Each guiding post 42 has an inclined surface 421 at an end thereof and an abutting end 422 at another end thereof. One guiding post 42 stands beyond an edge of the neck 40 instead of on a common surface where other three guiding posts 42 stand. A pair of first abutting projection 43 is formed on a top end of the pusher 4, and a pair of second abutting projections 43 is formed below the first abutting projections 43 and symmetric to each other. The first abutting projections and the second abutting projections 43 bias against the bugle portion 310 and below a bottom of the needle base 3. A dish 44 is formed on the pusher 4 and adjacent the lip 22 of the syringe body 2 for pushing the pusher 4. A drawing ring 45 is provided on the dish 44 for manual operation. The drawing ring 45 may be formed of various type, for example, one sheet, two sheets locking each other, or be formed of other shape for allowing a thumb therethrough.

The springy element 6 is formed between the syringe body 2 and the pusher 4 and may be a compressed spring. The springy element 6 has an end 60 for abutting against the lip 22 of the syringe body 2, and an opposite end 61 for abutting against the dish 44 of the pusher 4. A push sheet 24 is provided on an outer peripheral of the syringe body 2, whereby one hand may operate to retract the distorted needle. Namely, after injection, first finger and middle finger of one hand clamp the push sheet 24 and rotates left/right easily, and then rotates to retract the pusher 4 by approximate return force of the springy element 6, thereby pushing the needle base 3. Note that the springy element 6 and the push sheet 24 may be omitted as for an injection syringe with small diameter.

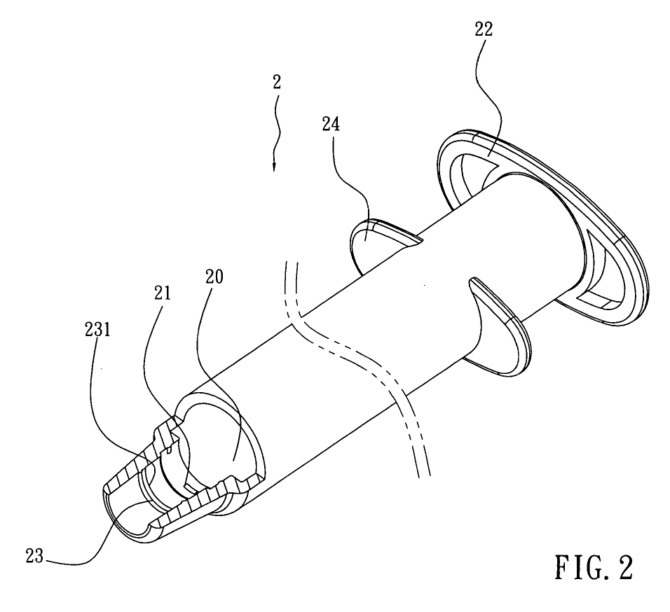

In assembly, with combination to FIGS. 1, 2, 3 and 7, the leakproof gasket 41 is assembled on the neck 40 of the pusher 4. The needle base 3 is mounted inside the syringe body 2. The springy element 6 is mounted on the pusher 4, and the pusher 4 is telescopically mounted in the chamber 20 of the syringe body 2. The needle base 3 displaces right/left relative to the syringe body 2 until the first stopper 23 and the second stopper 231 respectively abutting against the shoulder 34, as shown in FIG. 7. Finally, the needle 5 is assembled on the needle connecting portion 30 of the needle base 3.

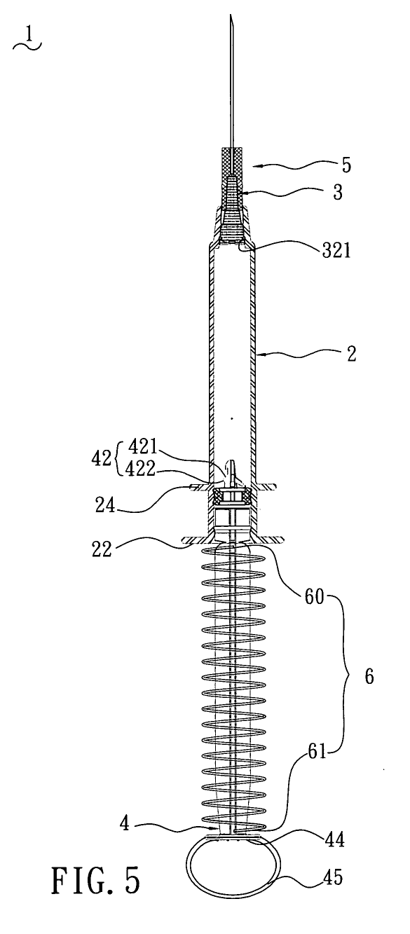

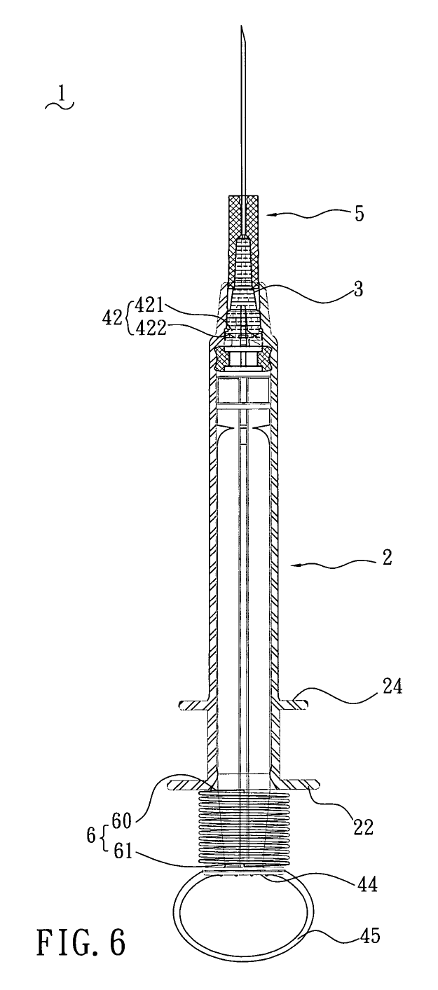

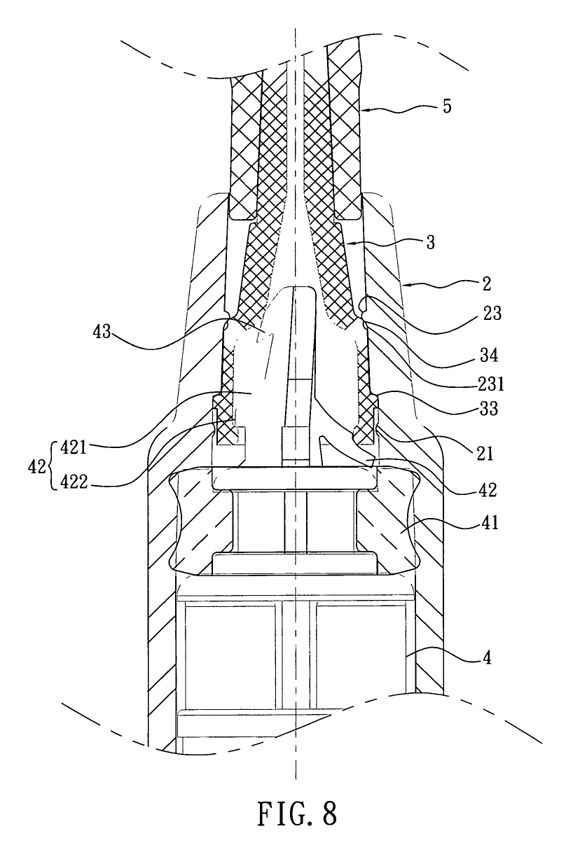

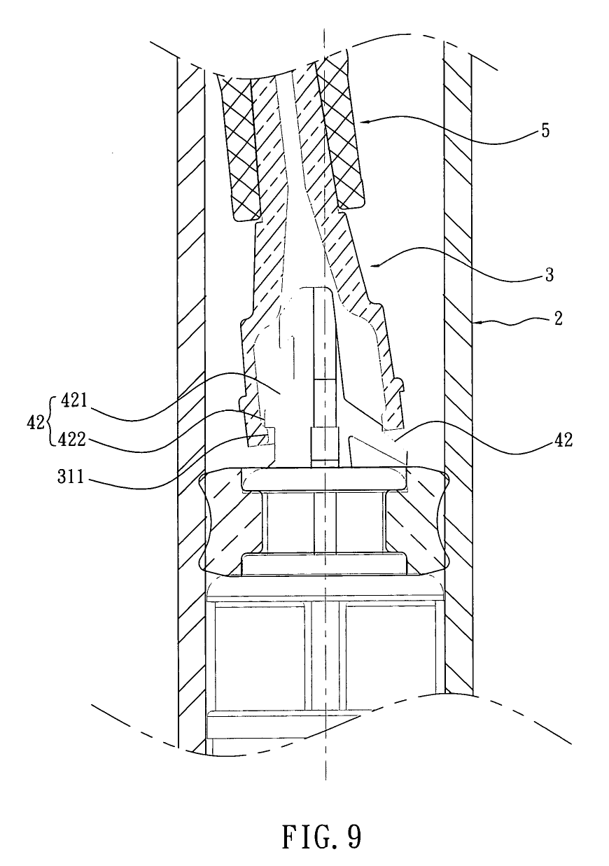

Referring to FIGS. 3, 4 and 5-8, when the pusher 4 is pushed for injection, the inclined surfaces 421 of the guiding posts 42 are guided by the guiding surfaces 321 of the ribs 32 of the needle base 3, and push medicine in the syringe body 2 forwardly, as shown in FIG. 6. During the pusher 4 moving, as shown in FIG. 7, the abutting projections 43 of the pusher 4 moves along the bugle portion 310, and are pressed by the bugle portion 310. After injection, a top end of the pusher 4 slightly deflects right and departs from a center line, as shown in FIG. 8. As shown in FIG. 9, the springy element 6 is pressed and therefore possesses energy.

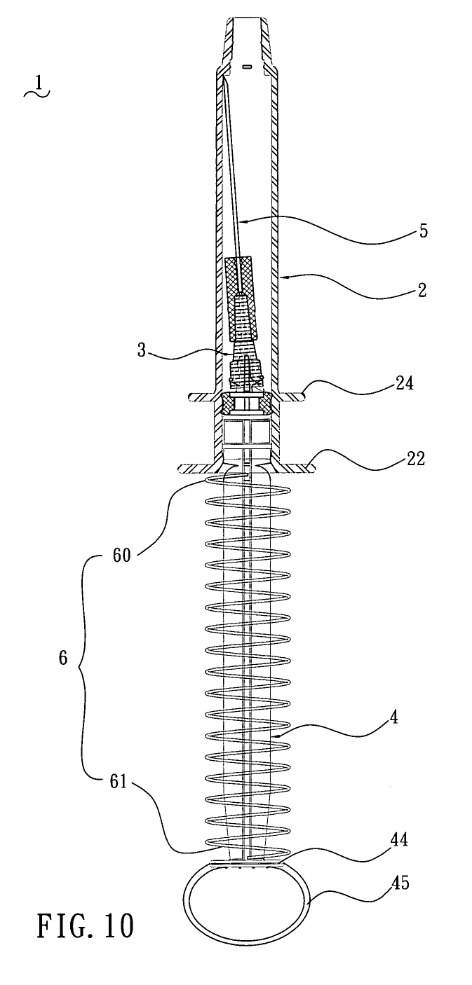

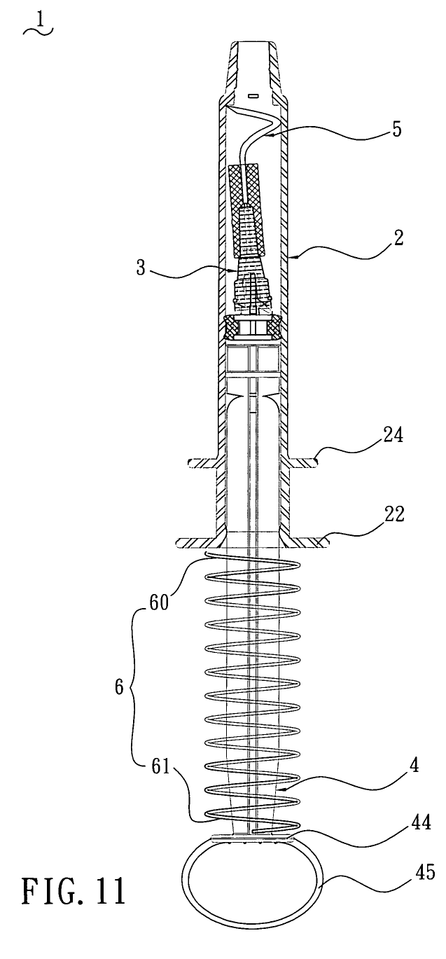

In order to draw out the pusher 4, as shown in FIG. 9, the needle base 3 rotates left/right to displace relative to the syringe body 2. The abutting surfaces 422 of the guiding posts 42 abut against the flange 311 of the needle base 3. With energy of the springy element 6 and the power of the pusher 4, the needle base 3 is retracted firmly. Meanwhile the needle base 3 is free. One guiding post 42 of the pusher 4 is slightly tilted, and further due to energy of the springy element 6, forming double deflection force for the pusher 4. Therefore, the needle base 3 is pushed aside and further deflects. Referring to FIGS. 10 and 11, one guiding post 42 beyond the neck 40 biases against a bottom of the needle base 3, and the tilted needle base 3 (in FIG. 10) pushes the pusher 4 forward and distorts the needle 5 (in FIG. 11).

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. An injection syringe adapted to assemble a needle thereon, comprising:
a syringe body being hollow and cylindrical, and defining a chamber therethrough, first protrusions being formed proximate an end of the syringe body and being distributed apart the same distance from each other, a lip being formed at an outer peripheral of an end of the syringe body opposite to the first protrusions;
a needle base assembled inside the syringe body and adjacent to the first protrusions, and including a needle connecting portion for connecting the needle, and a passage through two ends of the needle base, a bugle-shaped bugle portion extending and expanding far away from the needle connecting portion, at least two ribs being formed on an inner surface of the bugle portion, each rib having a guiding surface, a flange being formed at an end of the bugle portion, second protrusions being distributed radially on an outer surface of the needle base, the number of the second protrusions being equal to that of the first protrusions; and
a cylindrical pusher assembled inside the syringe body, and forming a neck on an end thereof adjacent to the first protrusions, a leakproof gasket being provided above the neck, a plurality of guiding post being formed on the neck, each guiding post having an inclined surface at an end thereof for abutting the flange of the needle base, and an abutting end at an opposite end thereof, at least an abutting projection being formed on a top end of the pusher for biasing against the bugle portion and below a bottom of the needle base, a dish being formed adjacent the lip of the syringe body for pushing the pusher.

2. The injection syringe as claimed in claim 1, further comprising a first stopper is integrally formed on an inner surface of the syringe body, and wherein a shoulder is formed on an outer surface of the needle base and fits to the first stopper.

3. The injection syringe as claimed in claim 2, further comprising a second stopper is integrally formed on the inner surface of the syringe body and below the first stopper.

4. The injection syringe as claimed in claim 3, wherein a recess is defined in a lower portion of the shoulder for corresponding to the second stopper.

5. The injection syringe as claimed in claim 4, wherein the first stopper and the second stopper have ring shape and integrally project from the syringe body for cooperating with the shoulder and the recess to prevent the pusher from excess movement and to facilitate the pusher disengaging, simultaneously to prevent leakage.

6. The injection syringe as claimed in claim 1, further comprising a springy element between the syringe body and the pusher, and a push sheet on an outer peripheral of the syringe body.

7. The injection syringe as claimed in claim 6, wherein the springy element is a compressed spring, and has an end abutting against the lip of the syringe body, and an opposite end abutting against the dish of the pusher.

8. The injection syringe as claimed in claim 1, further comprising a drawing ring on the dish.

9. The injection syringe as claimed in claim 1, wherein a portion of the needle base where the second protrusions are formed has a cylindrical shape.

10. The injection syringe as claimed in claim 1, wherein four first protrusions are spaced radially apart 90° from each other, and four second protrusions are spaced radially apart 90° from each other.

11. The injection syringe as claimed in claim 1, wherein four ribs are formed on an inner surface of the bugle portion and are radially spaced apart 90° therebetween.

12. The injection syringe as claimed in claim 1, wherein four guiding posts are radially spaced 90° apart therebetween, and wherein one guiding post stands beyond an edge of the neck instead of on a common surface where other three guiding posts stand.

13. The injection syringe as claimed in claim 1, wherein a first abutting projection is formed on a top end of the pusher, and a pair of second abutting projections are formed below and symmetric to each other.

14. The injection syringe as claimed in claim 10, wherein the first protrusion and the second protrusions are arcuate.
